(19) United States
(12) Patent Application Publication
(10) Pub. No.: US 2026/0365950 A1
(43) Pub. Date: Nov. 19, 2026

(54) APPARATUS AND METHODS FOR MODULATING MICROBIAL ACTIVITY

(71) Applicant: VERTUS ENERGY LIMIITED, Auckland (NZ)

(72) Inventor: Danilo Perez, Auckland (NZ)

(21) Appl. No.: 19/477,341

(22) PCT Filed: Apr. 17, 2024
(86) PCT No.: PCT/NZ2024/050041

§ 371 Date: Oct. 21, 2025

(30) Foreign Application Priority Data
Apr. 21, 2023 (NZ) 799195

(51) Int. Cl. C12M 1/107; C02F 11/04; C02F 103/20; C12M 1/12

(52) U.S. Cl. CPC C12M 21/04; C02F 11/04; C12M 25/10; C02F 2103/20

(57) ABSTRACT

An apparatus and methods for modulating microbial activity in organic waste streams for biogas production.

### FIELD OF THE INVENTION

The present invention broadly relates to apparatus and methods for modulating microbial activity in organic waste streams for biogas production.

### BACKGROUND TO THE INVENTION

Biogas is produced via the breakdown of organic matter in absence of oxygen, known as anaerobic digestion. The obtained gas is mostly composed of methane (CH₄) and carbon dioxide (CO₂), so it can be used as a fuel. Biogas is considered a low-emission fuel, and may play a fundamental role in decarbonisation of the energy matrix.

Anaerobic digesters generally comprise a chamber, an inlet for feedstock, an outlet for gas, which can be a vent or a gas recovery outlet, and an outlet for digestate. They can also comprise means for mixing the reactant liquor to promote digestion. Anaerobic digesters can be of significant size and may be formed of stainless steel, carbon steel, or concrete, including concrete cast in situ. Anaerobic digestion projects are usually large in scale, due to high capital costs and long payback periods. Existing approaches to anaerobic digestion suffer from long hydraulic retention times, and are vulnerable to operational fluctuations.

Microbial electrolysis cells (MEC) show promise in applications for improving yield in production of biogas, usually operating under mild conditions and starting from a wide range of organic matter feedstock. However, existing MEC configurations are not suitable for using at scale and under industrial conditions.

It is an object of the present invention to provide a sustainable waste-to-energy process that reclaims energy from waste by upgrading CO₂ to biomethane; to go at least some way to addressing one or more of the above disadvantages; and/or to at least provide the public with a useful choice.

Other objects of the invention may become apparent from the following description which is given by way of example only.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the invention provides a bioelectrode comprising a bacterial biofilm, wherein the bioelectrode has a porous structure with a range of pore sizes.

In a second aspect, the invention provides a microbial electrolysis cell (MEC) comprising a solid-state cathode; a solid-state anode; and an electro-active biofilm.

In a third aspect, the invention provides a process for colonizing a bioelectrode with microorganisms, comprising steps of: contacting an electrode with a mixture comprising microorganisms; and applying a potential to the electrode during the contacting step.

In a fourth aspect, the invention provides a process for anaerobic digestion of a feedstock in an anaerobic digester, the process comprising: providing a feedstock to a chamber of anaerobic digester to form a reactant liquor in the anaerobic digester; contacting the reactant liquor with a bioelectrode of a microbial electrolysis cell which comprises the bioelectrode and a counter electrode, and applying a potential to the reactant liquor.

In a fifth aspect, the invention provides an external module comprising a chamber, wherein the chamber is adapted for fluid communication with a chamber of an anaerobic digester.

In a sixth aspect, the invention provides an anaerobic digester comprising a bioelectrode according to the first aspect.

The following embodiments and preferences may relate alone or in any combination of any two or more to any of the above aspects.

In some embodiments, the bioelectrode comprises primary pores in a first size range, and secondary pores in a second size range.

In some embodiments, the bioelectrode comprises a biocompatible material and/or a carbon-based material, for example felts, papers, sintered, porous material of either carbon, metallic or plastic base.

In some embodiments, the electrode comprises fibre strands. In some embodiments, the fibres form a 3D open structure of primary pores. In some embodiments, the secondary pores are provided as indentations. In some embodiments, the indentations are formed by a pre-treatment of the fibres. Optionally, the pre-treatment comprises applying a potential to the electrode.

In some embodiments, the electrode surface is functionalised by immobilized microbes, enzymes, or a catalytic material.

In some embodiments, the electrode has a surface area of at least 1 m²/g, more preferably at least 2 m²/g.

In some embodiments, the biofilm is a mature biofilm.

Preferably, the biofilm comprises at least one methanogenic microorganism; and at least one electroactive microorganism.

In some embodiments, the MEC of the second aspect comprises a bioelectrode according to the first aspect.

In some embodiments, the MEC further comprises a dielectric material between the cathode and the anode.

In some embodiments, the potential applied during the step of contacting an electrode with a mixture comprising microorganisms is about +1.5 V/SCE.

In some embodiments, the potential is applied during the contacting step for a period of at least about 1 hour, preferably at least about 2 hours, more preferably for about 2 hours. While a shorter time period can be used to facilitate microbial attachment, the time period can optionally be longer, for example up to about 8 hours or longer.

In some embodiments, the potential applied during the contacting step is an anodic potential, and the microorganisms which colonize the bioelectrode comprise methanogenic microorganisms.

In some embodiments, the process further comprises a pre-anodisation step of applying an anodic potential to the electrode prior to the contacting step. In some embodiments, the pre-anodisation step comprises applying a potential of about +1.5 V/SCE.

In some embodiments, the potential is applied to the electrode in cycles during the pre-anodisation step. In some embodiments, the cycles are of a period of about 15 min on, 15 min off. Optionally, successive cycles can be applied over a period of about 24 hours.

In some embodiments, the bioelectrode of the microbial electrolysis cell is positioned within the chamber of the anaerobic digester.

In preferred embodiments, the bioelectrode comprises a biofilm.

In various embodiments of the second or further aspects, the bioelectrode is a bioelectrode according to the first aspect. In some embodiments, the bioelectrode is a cathode. The cathode may optionally be adapted to favour direct methanogenesis, or adapted to favour direct interspecies electron transfer. In such embodiments the cathode may optionally comprise a material such as graphite felt, or stainless steel, as described herein.

In some embodiments, the bioelectrode is separated from the counter electrode by a dielectric material.

In some embodiments, the anaerobic digester comprises a microbial consortium comprising planktonic microbes and the bioelectrode of the microbial electrolysis cell comprises a microbial biofilm.

In some embodiments, the microbial electrolysis cell is positioned inside the chamber of the anaerobic digester. In other embodiments, the microbial electrolysis cell is positioned outside the chamber of the anaerobic digester, and is in fluid communication with the chamber of the anaerobic digester.

In some embodiments, the voltage applied between the bioelectrode and the counter electrode during the anaerobic digestion process is greater than 100 mV, or greater than 300 mV, or greater than 600 mV. Preferably, the voltage applied between the bioelectrode and the counter electrode is in the range 600 mV to 1200 mV, more preferably in the range 800 mV to 1000 mV.

In some embodiments, the temperature in the anaerobic digester is within the range from about 30 to about 45 °C, preferably from about 39 to about 42 °C, or from about 35 to about 39 °C, more preferably about 38 °C.

Optionally, the feedstock/reactant liquor comprises municipal wastewater and/or manure, preferably with a total solids content between 1 and 10%, more preferably between 1 to 5% or 5 to 7%.

In some embodiments, the chamber of the external module is adapted to receive a bioelectrode according to the first aspect.

In some embodiments, the chamber of the external module further comprises a bioelectrode according to the first aspect.

In some embodiments, the bioelectrode of the first aspect was produced by a process according to the third aspect.

In some embodiments, the external module further comprises a pump for exchanging fluid between the chamber of the external module and the chamber of the anaerobic digester.

In some embodiments, the external module is adapted to be stackable with another external module.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The present invention will be described with reference to the accompanying figures, in which:

FIG. 1 shows a reactor according to a first embodiment of the invention with an internal MEC;

FIG. 2 shows a reactor according to a second embodiment of the invention with an external MEC;

FIG. 3 is a graph showing the average biogas production (L/d; bars) and methane content (percentage; circles) for an anaerobic digester (AD) reactor, and reactors having an internal external MEC (ModIn), and external MEC (ModEx) over a six-week period;

FIG. 4 is a graph comparing methane production rate (mL CH₄/L (reactor)/d) for an anaerobic digester (AD) reactor, and reactors having an internal external MEC (ModIn), and external MEC (ModEx);

FIG. 5 is a graph comparing chemical oxygen demand removal efficiency (% COD removal) for an anaerobic digester (AD) reactor, and reactors having an internal external MEC (ModIn), and external MEC (ModEx);

FIG. 6 is a graph comparing methane conversion rate (MCR) for an anaerobic digester (AD) reactor, and reactors having an internal MEC (ModIn), and external MEC (ModEx);

FIG. 7 is a plot of increase in surface area over time when cycles of 15 mins On/Off of 1.5 V are applied to an anode of graphite felt;

FIG. 8 is a plot of increase in surface area over time for two electrodes of graphite felt in Fe₂SO₄ electrolyte of 4.3 mg/L (low) and 430 mg/L (high) concentration respectively;

FIG. 9 is a graph comparing methane production rate (MPR) for an anaerobic digester (AD) reactor, and reactors having an internal MEC (ModIn), and external MEC (ModEx), with graphite felt or stainless steel cathodes, at open circuit potential (OCP), using a full cell potential control strategy;

FIG. 10 is a graph comparing methane production rate (MPR) for reactors having an internal MEC (ModIn), and external MEC (ModEx), with graphite felt or stainless steel cathodes, at a potential of 0.8 V, using a full cell potential control strategy; and

FIG. 11 is a graph comparing methane production rate (MPR) for reactors having an internal MEC (ModIn), and external MEC (ModEx), with graphite felt or stainless steel cathodes, at a potential of 1.5 V, using a full cell potential control strategy.

### DETAILED DESCRIPTION OF THE INVENTION

### Definitions

The following definitions are presented to better define the present invention and as a guide for those of ordinary skill in the art in the practice of the present invention. Unless otherwise specified, all technical and scientific terms used herein are to be understood as having the same meanings as is understood by one of ordinary skill in the relevant art to which this disclosure pertains.

The general chemical and biological terms used herein have their usual meanings.

Examples of definitions of common terms in microbiology, molecular biology and biochemistry can be found in Methods for General and Molecular Microbiology, 3^{rd} Edition, C. A. Reddy, et al. (eds.), ASM Press, (2008); Encyclopedia of Microbiology, 2nd ed., Joshua Lederburg, (ed.). Academic Press, (2000); Microbiology By Cliffs Notes, I. Edward Alcamo, Wiley, (1996); Dictionary of Microbiology and Molecular Biology, Singleton et al. (2d ed.) (1994); Biology of Microorganisms 11th ed. Brock et al., Pearson Prentice Hall, (2006); Biodiversity of Fungi: Inventory and Monitoring Methods, Mueller et al. Academic Press, (2004); Genes IX, Benjamin Lewin, Jones & Bartlett Publishing, (2007); The Encyclopedia of Molecular Biology, Kendrew et al. (eds.), Blackwell Science Ltd., (1994); and Molecular Biology and Biotechnology: a Comprehensive Desk Reference, Robert A. Meyers (ed.), VCH Publishers, Inc., (1995).

The term “comprising” as used in this specification and claims means “consisting at least in part of”. When interpreting each statement in this specification and claims that includes the term “comprising”, features other than that or those prefaced by the term may also be present. Related terms such as “comprise”, “comprised” and “comprises” are to be interpreted in the same manner.

As used herein the term “and/or” means “and” or “or”, or both.

As used herein “(s)” following a noun means the plural and/or singular forms of the noun.

In one embodiment the term “statistically significant” as used herein refers to the likelihood that a result or relationship is caused by something other than random chance. A result may be found to be statistically significant using statistical hypothesis testing as known and used in the art. Statistical hypothesis testing provides a “P-value” as known in the art, which represents the probability that the measured result is due to random chance alone. It is believed to be generally accepted in the art that levels of significance of 5% (0.05) or lower are considered to be statistically significant.

The term “microbial consortium” as used herein refers to a group of two or more species of microorganisms. Microorganisms are present in the planktonic state, and also attached to the bioelectrode(s). In some embodiments, the microorganisms form a biofilm on the bioelectrode(s). The microbial consortium is not homogeneous throughout the reactor and electrodes, as some microorganisms are more suited to colonising the cathode, some are more suited to colonising the anode, and some remain in the planktonic state.

### Anaerobic Digester

As used herein, “anaerobic digester” refers to a vessel in which anaerobic digestion takes place. While industrial anaerobic digesters are very large, smaller vessels which might be suitable for a domestic premises are also encompassed within the term anaerobic digester as used herein. The reactor vessels described in the Examples below are referred to herein as “reactors”, but can also be considered to be “anaerobic digesters” as that term is used herein.

An industrial anaerobic digester can have a working volume in the range on the order of up to about 10,000 m³, and larger capacities can be reached by connecting several smaller reactors in parallel. An anaerobic digester suitable for a domestic premises is smaller and can have a working volume as small as 1 m³, while an anaerobic digester on a farm or agricultural premises might be in the range 100-200 m³ or greater. Commercial anaerobic digesters for producing biogas can be for example 100-1000 m³, a common size having a working volume of about 600 m³. The invention described herein is suitable for use in a reactor having a working volume in any of these size ranges.

Anaerobic digestion has four stages; hydrolysis, acidogenesis, acetogenesis and methanogenesis, each associated with different microbial species. Anaerobic digestion processes include batch processes, and continuous processes. In a batch process, the four stages occur sequentially. Batch processes are typically slower and require larger volumes to achieve production similar to continuous processes. The present invention is suited to either a batch process or a continuous process.

In an aspect, the invention relates to an anaerobic digester comprising an MEC as described herein. The MEC comprises at least a cathode and an anode. In the anaerobic digester of the invention, the MEC can be provided in various ways. In some embodiments, the working electrode of the MEC is positioned within the chamber of the anaerobic digester and the counter electrode of the MEC is positioned within the chamber of the anaerobic digester. In other embodiments, the working electrode of the MEC is positioned within the chamber of the anaerobic digester and the counter electrode of the MEC forms a part of the wall of the chamber. In other embodiments, the working electrode and the counter electrode of the MEC is positioned outside the chamber of the anaerobic digester, and are in fluid communication with the chamber of the anaerobic digester, for example by an arrangement of an external module as described herein. This provides for a simple means of retrofitting an existing anaerobic digester as an MEC anaerobic digestion system.

Reaction rates can be enhanced by mixing the feedstock improving transport of the reactants between the bulk of the feedstock and the surface of the cathode and/or anode. For example, means to mix the reactant liquor may comprise a pump and circulation line, an impeller, baffles, deflector(s), rotor/stator pair(s), propeller(s), stirrer(s), screw(s), or other mixing means or geometric features of the chamber or external module, such as features related to the inlet(s) or outlet(s).

In some embodiments, the cathode and/or anode can form part of or be associated with one or more blades of an impeller or other mixing means.

In some embodiments, the cathode and/or anode can form part of or be associated with one or more baffles inside the chamber.

In some embodiments, the cathode and/or anode can be positioned in proximity with one or more blades of an impeller or other mixing means, or an inlet or outlet of a pumped circulation line, or one or more baffles inside the chamber of the anaerobic digester, or one or more baffles inside the chamber of the external module.

### Feedstock

The invention may utilize a feedstock of organic waste. The feedstock can be an organic feedstock can comprise organic waste or an organic waste stream, such as municipal wastewater, manure, food scraps, biosolids, food scraps, spent grain, or yeast. Once in the anaerobic digester, digestion of the feedstock commences and the composition changes. At the digestion stage, the feedstock is referred to as “reactant liquor”. The reactant liquor is termed a digestate once it leaves the anaerobic digester. The digestate can be used as a fertiliser.

In some embodiments the feedstock undergoes a pre-treatment or conditioning step, typically including temperature adjustment, particularly where legal requirements dictate a heat treatment of the feedstock for sanitary reasons.

Optionally, a further pre-treatment and conditioning step can be carried out, such as adjustment of water content, for example by diluting with water; or reduction of particle size distribution of solids, for example using a hammermill or grinder pumps.

When the lignocellulosic content of the waste is high, an addition of a co-inoculum to the feedstock such as zebra manure, horse manure, or another organic fertiliser containing a high concentration of lignocelluloses, is recommended. The co-inoculum is preferably heterogeneous in composition, size and structure, and can be easily degraded by enzymes or bacteria.

### Microbial Electrolysis Cell (MEC)

The MEC comprises a solid-state cathode; a solid-state anode, and an electro-active biofilm. In some embodiments, the MEC further comprises a reference electrode. The cathode and anode are connected to one or more power supplies or a potentiostat. In use, the MEC is immersed in a conductive electrolyte in the presence of the microorganisms and reactant liquor.

Optionally, the MEC further comprises a dielectric material. Connections between the anode, cathode, and reference electrode if present can suitably be made via electrical connectors comprising a corrosion-resistant material.

At least one of the cathode and/or the anode of the MEC comprises an electro-active biofilm.

In some embodiments, the anode comprises an anodic biofilm comprising a first group of microorganisms, and the solid-state electrode. As discussed herein, the first group of microorganisms can be developed on the electrode. Optionally, the anode can further comprise a mechanical support where the biofilm can form.

In some embodiments, the cathode comprises a cathodic biofilm comprising a second group of microorganisms, and the solid-state electrode. As discussed herein, the second group of microorganisms can be developed on the electrode. Optionally, the cathode can further comprise a mechanical support where the biofilm can form.

Installation of the MEC as an internal unit in an anaerobic digester must accommodate the feedstock inlet, waste and off-gas flow outlets, and any heating, monitoring or sampling apparatus already present in the anaerobic digester, while also providing the required electrical connections and with regard to maintenance access. The configuration of the MEC is also dictated by the electrodes' size, and must address flow dynamics, mixing, and encouraging contact between the electrodes and reactant liquor, for example using an impeller or an arrangement of baffles as discussed herein.

### Physical Arrangement Between the Cathode and Anode

In some embodiments, the cathode and anode can suitably be arranged with respect to each other to reduce ohmic overpotential due to internal electrical resistance. For example, decreasing the distance between the electrodes can reduce the ohmic overpotential.

In some embodiments, the cathode and anode can suitably be arranged with respect to each other to encourage the fermentation liquor through the anaerobic digester or external module. In some embodiments, the cathode and anode can suitably be arranged with respect to each other to minimize the shear forces acting in the digestate. This can be achieved for example by modelling the interior of the AD using computational fluid dynamics.

In some embodiments, the cathode and anode can suitably be arranged with respect to each other to provide a suitable biofilm growing space. For example, the biofilm can grow up to 2 cm in thickness. The electrodes should be sufficiently spaced to avoid contact between the biofilms growing on the electrodes. Preferably, the electrodes are more than 2 mm apart. In some embodiments, the electrodes are 2-50 mm apart, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 50 mm apart.

Direct contact between the cathode and anode should be avoided, as the digestate would then not act as an electrolyte. Thus in some embodiments, the cathode and anode are separated by a dielectric material.

### Dielectric Material

The dielectric material comprises a non-conductive insulator to avoid direct contact between electrodes. It may be made from polyester e.g. a stretched polyester film such as biaxially-oriented polyethylene terephthalate (BoPET) (for example Mylar™), or polypropylene. Alternatively polytetrafluoroethylene (for example Teflon™), non-woven materials or other plastics may be used.

### Electrode Architecture/Design of the Electrode

For durability, the electrodes are preferably solid-state rather than gel or electrolyte. The connector(s) can suitably comprise corrosion resistant materials, for example titanium, stainless steel, graphite sheets, carbon felts, or coated metals. To reduce energy consumption, the material of the electrodes is preferably chosen to keep to a minimum the ohmic and activation overpotentials.

In some embodiments, the bioelectrode comprises a biocompatible material and/or a carbon-based material, for example felts, papers, sintered, or porous material of carbon, metallic or plastic base.

The surface of the electrode can advantageously be provided with a porous structure, having a range of pore sizes. In some embodiments, the surface of the electrode comprises primary pores in a first size range, and secondary pores in a second, smaller size range. The primary pores can be provided as a three-dimensional (3D) open structure. This provides sites of attachment for electroactive microorganisms and improves mass transport. Smaller, secondary pores increase surface area, promoting direct methanogenesis.

Increasing the range of pores sizes in the open 3D structure with provides increased surface area, with smaller pores useful for microbial colonization and larger pores mitigating clogging.

In some embodiments, the electrode comprises fibre strands and the fibre strands form a 3D open structure of primary pores. For example, the spaces between fibre strands may be on the order of about 10 μm in size or greater, allowing for movement of the microorganisms through the electrode. The secondary pores can be formed by a treatment of the fibres. In some embodiments, the secondary pores are formed by applying an anodic potential to the electrode (pre-anodization step). “Secondary pores” as described herein, can also refer to surface indentations. Indentations in the surface of the fibres of a size about 1 μm are comparable in size to a microbial cell. They may provide an increase in surface area, and improve the roughness necessary for microbial attachment.

Optionally, the fibres or the electrode surface can be treated to facilitate microbial attachment, for example using thermal treatment, or plasma or etching techniques.

Optionally, the electrode surface is functionalised by immobilized microbes, enzymes, or other catalytic materials, for example titanium which has a catalytic characteristic for hydrogen production.

Preferably, the electrode has a surface area of at least 1 m²/g, more preferably at least 2 m²/g, for example at least 3 m²/g or at least 4 m²/g of electrode.

Pre-treatment of the electrode to form a biofilm is discussed further below.

In some embodiments the electrode comprises a material which favours direct methanogenesis, such as stainless steel or graphite plates. In other embodiments, the electrode comprises a material which favours direct interspecies electron transfer (DIET), such as carbon felt.

In some preferred embodiments the electrode can be chosen with reference to the feedstock which will be digested. Depending on their makeup, different feedstocks reach a bottleneck at different stages of the anaerobic digestion process. For example, manure contains a high content of lignocellulosic biomass, the bottleneck stage is the hydrolysis stage, so the choice of a GF electrode to encourage hydrolytic microorganisms may be considered. Municipal wastewater or food scraps is more easily hydrolysed and the bottleneck is at the final methanogenesis stage, in which case a GF cathode, despite being more expensive than a stainless steel cathode, may be chosen to encourage methanogens. In addition, feedstock containing solids materials would be more likely to foul a porous electrode, so non-porous electrodes may be considered.

### Microorganisms

The invention uses a microbial consortium comprising one or more microorganisms selected from the operational taxonomic units (OTU): hydrolytic microorganisms, acidogenic microorganisms, acetogenic microorganisms, methanogenic microorganisms, and preferably comprising both acetolactic microorganisms and hydrogenotrophic microorganisms.

While the hybrid consortium implies the co-existence of both a biofilm and planktonic cells, the OTU are not evenly distributed between the biofilm and planktonic cells, but rather, are associated with the environment more appropriate for their metabolism. Those microbial species which are active in the oxidative stages of the anaerobic digestion (hydrolysis, acetogenesis) tend to prefer the anode, whereas methanogenic microorganisms are active in the methanogenic stage associated with the cathode.

The cathode is a source of electrons for methanogenesis, but methanogenic microorganisms are negatively charged and do not interact with the cathode due to electrostatic repulsion. Other microbial species can transfer electrons from the cathode to the methanogenic microorganisms via direct interspecies electron transfer (DIET).

The microbial consortium can include methanogenic microorganisms including archaea which can be obtained from animal manure, electroactive microorganisms capable of interacting electrically with solid-state electrodes, and microorganisms capable of performing direct interspecies electron transfer, for example transfer of electrons between the electroactive microorganism the methanogenic microorganisms, and microorganisms which produce extracellular polymeric substances (EPS) such as exopolysaccharides which can act as an anchor to form a biofilm. Optionally, the microbial consortium can comprise microorganisms which fulfil more than one of these roles. In some embodiments, the various species of the microbial consortium can be obtained from animal manure.

A colony-forming unit (CFU) is a unit used to estimate the number of viable bacteria in a sample. Determining colony-forming units requires culturing the microbes and counts only viable cells, i.e., those able to multiply and form a visible colony.

### Pre-Colonisation of the Electrode

Before or during operation of the anaerobic digester, a biofilm forms on the cathode and anode, thereby forming a bioelectrode. This process may be enhanced by application of a potential to the electrode, drawing electroactive microorganisms towards the electrode by electrostatic attraction.

In some embodiments a biofilm, preferably a mature biofilm, is formed on the electrode as a pre-treatment prior to its use in the anaerobic digestion system. Preferably, the biofilm comprises at least one methanogenic microorganism; and at least one electroactive microorganism.

A mature biofilm acts as a natural defence mechanism against environmental fluctuations including pH variation and presence of toxins increasing the overall resilience of the system.

By controlling the polarity of the solid-state electrodes, it is possible to control its function as donor/acceptor of electrons and promote the maturation of biofilm over specific electrodes.

Thus, in an aspect, the invention provides a process for colonizing a bioelectrode with microorganisms, comprising steps of contacting an electrode with a mixture comprising microorganisms, and applying a potential to the electrode during the contacting step.

The potential applied is preferably greater than 300 mV, and lower than 2000 mV, more preferably in the range 600 mV to 1200 mV, even more preferably in the range 800 to 1000 mV.

In some embodiments, the bioelectrode is for use as a biocathode, and the potential applied is an anodic potential. That is, the bioelectrode acts as an anode during the colonizing step. The anodic potential attracts electroactive microorganisms which would not be attracted to a cathode. Optionally, prior to the colonizing step, the process comprises a step of applying an anodic potential to the electrode to form secondary pores (the pre-anodization step discussed above).

The biofilm generally takes some time to form on the electrode, typically on the order of days.

In some embodiments the presence of the biofilm on the electrode can be detected by a change in current.

Pre-colonisation of the biofilm onto the electrode provides advantages where the intended feedstock does not have the appropriate microbial flora for anaerobic digestion. For example, methanogenic bacteria are anaerobic and sensitive to oxygen, and are not present in municipal wastewater in enough quantity to effectively colonize the electrode. A high proportion of co-inoculum is typically used in prior art anaerobic digestion processes, for example up to 10 kg manure: 90 kg food scraps. Pre-seeding of the biofilm onto the electrode avoids the need for large quantities of co-inoculum.

### Anaerobic Digestion Process

In an aspect, the invention relates to a process for anaerobic digestion of a feedstock in an anaerobic digester, the process comprising:
providing a feedstock to a chamber of anaerobic digester to form a reactant liquor in the anaerobic digester;
contacting the feedstock with a bioelectrode of a microbial electrolysis cell (MEC) which comprises the bioelectrode and a counter electrode, and
applying a potential between the bioelectrode and counter electrode.

Steady-state operation of the anaerobic digestion process can usually be achieved after about three hydraulic-retention time (HRT) periods. For example, if the HRT is 20 days, steady-state operation can be reached after about 60 days.

The process of the invention uses a low voltage to enhance the overall methane production rate. This reduces the hydraulic retention time (HRT) required to complete the reaction, therefore reducing the size of the system required to handle a given feedstock, and reducing costs of operating the system.

The process of the invention has increased resilience to operational fluctuations due to the mature electrobiofilm formation, which is a natural formation that decouples the cellular retention time from the hydraulic retention time-increasing the microbial population in the system.

A lower operating temperature will require less energy consumption for heating, but will have a reduced reaction rate. In some embodiments the operating temperature range is from about 30 to about 45 °C, for example about 35 to about 45 °C, about 35 to about 42 °C, about 38 to about 42 °C, or about 35 to about 40 °C. Preferably the operating temperature range is from about 39 to about 42 °C, or from about 35 to about 39 °C.

### External Module

In an aspect, the invention relates to an external module comprising a chamber, wherein the chamber is adapted for fluid communication with a chamber of an anaerobic digester.

Advantageously, the external module can be retrofitted existing anaerobic digesters. It also provides for ease of substitution of the MEC, ease of maintenance of the MEC, and ease of coupling multiple MEC to a single anaerobic digester. Thus, the external module provides a “plug-and-play” solution to enhance performance and operation of anaerobic digesters.

The external module comprises an inlet and outlet to allow reactant liquor to pass through, the required electrical connections, and can optionally include a port or opening for maintenance access, and sensor(s) to monitor the digestion process. The external module is also configured to accommodate the total surface area of the electrodes.

### Applied Potential

The electrical voltage or potential applied by the power supply, power supplies or potentiostat should be such to overcome the ohmic, diffusion and activation overpotentials to ensure that the nutrients and reagents effectively reach the reaction site, allowing the electroactive biofilm to seize the electron donor/acceptor capacity of the relevant electrode.

The voltage may be applied as either a total cell potential, or as a half-cell potential versus a reference electrode. Using a total cell potential control strategy, only two electrodes (working and counter electrodes) work for both potential difference control and as electrical circuit for conducting the resulting current, i.e. the potential between the two half cells. Using a half-cell potential control strategy, the anode or cathode is connected to a reference or pseudo-reference electrode, and a controller is used to maintain the voltage between the working electrode and the reference or pseudo-reference electrode, with a minimal current flow through it and directing it to the counter electrode.

The voltage applied is preferably greater than 300 mV, and lower than 2000 mV, more preferably in the range 600 mV to 1200 mV, even more preferably 800 mV to 1000 mV.

Optionally, the voltage can be applied intermittently, and for example can use a renewable source of energy which may be fluctuant, for example photovoltaic panels or wind power.

### Example 1—Anaerobic Digestion Apparatus with Internal MEC

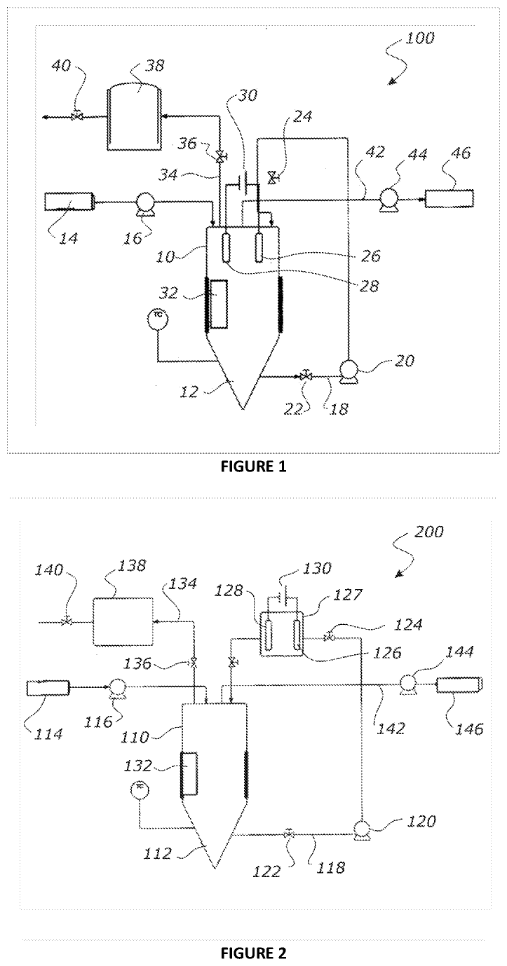

FIG. 1 shows a general schematic for an anaerobic digester 100 which comprises a reactor having a body 10 enclosing a reaction chamber 12. The reactor is provided with a source of feedstock 14, which is supplied to the reaction chamber 12 via a pump 16. The feedstock is an organic feedstock as described herein. The pump 16 can be a macerating pump, peristaltic pump, progressive cavitation pump, centrifugal pump or other pump suitable for feeding the feedstock to the reaction chamber 12.

Once in the reaction chamber 12, the feedstock (termed reactant liquor) remains in the reaction chamber for a time sufficient for anaerobic digestion to take place (the hydraulic retention time of the feedstock as discussed herein). In the example shown, the feedstock can be circulated within the reaction chamber 12 via a circulation line 18 and pump 20. Valves 22 and 24 control use of circulation line 18.

While circulation line 18 improves circulation of the feedstock, in embodiments of the invention with the electrodes internal to the reaction chamber 12, circulation line 18 is not required. Optionally, alternative means for circulating the reactant liquor within the reaction chamber can be provided, for example impellers or baffles as discussed herein.

An MEC is provided in the form of an anode 26 and cathode 28, connected by a power source 30. The temperature is maintained by a heater 32 and controlled by a temperature control TC.

Gases produced in the reaction chamber 12 collect in the headspace of the reaction chamber and exit the via a gas line 34 controlled by a valve 36. They are then collected in a receptacle 38 which can for example be a gas bag. A valve 40 provides for management of the gases. Reactant liquor exits the reaction chamber via exit line 42 controlled by pump 44, and is then termed digestate, being received in digestate storage 46. It will be appreciated that the replacement of the feedstock in the reaction chamber could be achieved using only one of pumps 16 and 44. In the present example the feedstock was replaced at a rate of 1 L/day, corresponding to a replacement rate for the feedstock of about 5% of the working volume of the reaction chamber per day.

### Example 2—Anaerobic Digestion Apparatus with External MEC Module

FIG. 2 shows a general schematic for an anaerobic digester 200 which comprises a reactor having a body 110 enclosing a reaction chamber 112. The reactor is provided with a source of feedstock 114, which is supplied to the reaction chamber 112 via a pump 116. The feedstock is an organic feedstock as described herein. The pump 116 can be a macerating pump, peristaltic pump, progressive cavitation pump, centrifugal pump or other pump suitable for feeding the feedstock to the reaction chamber 112. The temperature in the reaction chamber 112 is maintained by a heater 132 and controlled by a temperature control TC.

Once in the reaction chamber 112, the reactant liquor remains in the reaction chamber for a time sufficient for anaerobic digestion to take place (the hydraulic retention time of the reactant liquor as discussed herein). The reactant liquor is circulated within the reaction chamber 112 via a circulation line 118 and pumps 120, 131, controlled by valves 122, 124, and 131. As shown in the figure, after passing through circulation line 118 and pump 120, the reactant liquor enters external module 127. An MEC is provided in external module 127, comprising an anode 126 and cathode 128, connected by a power source 130.

Gases produced in the reaction chamber 112 collect in the headspace of the reaction chamber and exit the via a gas line 134 controlled by a valve 136. They are then collected in a receptacle 138 which can for example be a gas bag. A valve 140 provides for management of the gases. Reactant liquor exits the reaction chamber via exit line 142 controlled by pump 144, and is then termed digestate, being received in digestate storage 146. It will be appreciated that the replacement of the feedstock in the reaction chamber could be achieved using only one of pumps 116 and 144. In the present example the feedstock was replaced at a rate of 1 L/day, corresponding to a replacement rate for the feedstock of about 5% of the working volume of the reaction chamber per day.

### Example 3—Anaerobic Digestion with Internal MEC and External MEC Module

### Materials and Methods

A series of reactors were operated with two graphite felt electrodes provided to the internal volume of the reactor, or as an external module, as outlined below. The electrodes were prepared in a similar manner to that described in Example 5 below. The reactors were stainless steel vessels having a 30 L total volume, giving a 20 L working volume.

The reactors operated continuously circulating the content at a flow rate of 2 L/min using a peristaltic pump and circulation line controlled by a timer configured to provide a cycle of 15 min On, 15 min Off.

### Conditions and Replicates, Feedstock

The feedstock used in the examples below was liquid cow manure, which had been pre-treated to remove solid particles. The treatment comprised mixing solid cow manure with an equivalent mass of fresh water, stirring to disaggregate the manure with a hand drill and Rushton impeller, and leaving overnight. The mixture was then stirred further to disaggregate, and filtered to remove solid particles using a nylon sleeve with pores of about 0.5 mm. A further equivalent mass of water was added. This process was repeated until a ratio of 1:5 kg of initial manure:water had been added, producing a liquid manure.

The pre-treatment to produce liquid manure avoids scaling-down issues in the relatively small working volume of the reactors used in this example. The solid material removed from the manure has a high lignocelluosic content and does not contribute significantly to biogas production. The lignocellulosic content contributes to the chemical oxygen demand (COD) of the material which includes the total organic matter, but not to the biological oxygen demand (BOD). These solids also represent an operational challenge in existing anaerobic digestion processes.

Table 1 lists the variables monitored for each batch of liquid manure. The manure to water ratio can be adjusted as necessary to standardise between batches.

In this example, the ratio of manure to water used is 1:1, as the estimated dilution rate found in a typical dairy cow farm in New Zealand, where it is collected with water before pumping it to the paddocks, pond, or corresponding treatment. The test conditions are not designed to be favourable for the inventive process, but rather as representative conditions that can be found in a ‘typical’ anaerobic digestion plant.

TABLE 1
Manure characterization

| | Stored | g/L | | | | | BOD/ | TSS/ |
|---|---|---|---|---|---|---|---|---|
| Sample | days | BOD | COD | TS | VS | TSS | COD | TS |
| | 0 | 2.28 | 18.10 | 12.39 | 8.98 | 7.28 | 13% | 59% |
| Manure | 2 | 1.82 | 9.35 | 18.48 | 13.02 | 12.25 | 19% | 66% |
| Average | | 1.92 | 11.99 | 11.11 | 7.95 | 6.81 | 17% | 58% |

The reactors operated under the same constant conditions and were fed with the standardised liquid manure. Peristaltic pumps were used for harvesting and feeding to achieve a throughput of 1 L/day, giving a constant volume of about 20 L maintained in the reactor with a hydraulic retention time (HRT) of 20 days. Automatic temperature controllers in the reactors were set to 38.5 °C, turning on when the reactant liquor fell below 38 °C, and the off gas collected in a gas bag. pH was actively controlled to be maintained in a specific range (7.5 to 8, by adding NaOH when necessary).

In some of the reactors, the electrodes were incorporated in the main reaction vessel (ModIn 1, 2, 3, 4). As discussed herein, in response to the engineering challenges of carrying out this process at an industrial scale, some tests employed an external module (ModEx 1, 2, 3, 4). Eight reactors were operated for the example, with four replicate reactors being operated for each of the ModIn and ModEx configurations. Additionally, a ninth reactor was operated without electrodes, to model the performance of a typical anaerobic digester (AD). In the ModIn configuration, the electrochemical cell is immersed within the main reaction vessel, whereas the ModEx configuration has an external module, the content of which is continuously exchanged through the main reaction vessel.

### Results

### Gas Production Performance

As the organic loading rate (OLR) was increased, the system went through a transient period of increasing methane production rate (MPR) before day 30. The AD control had the lowest MPR, mostly due to lower biogas production, although the methane content is also in the lower end of the group, despite being higher than expected from literature references.

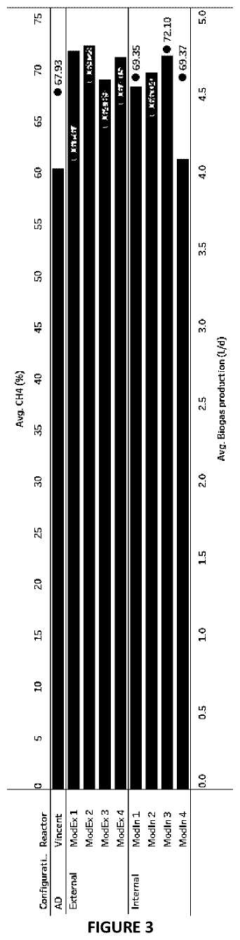

FIG. 3 shows the average biogas volumetric production and the methane content for the whole six-week period.

Industrial applications are more commonly concerned about the biogas production because the methane content usually is traditionally determined by the waste used as feedstock, and a certain quality of the feedstock and of gas is assured by including required pre/post-treatment, accepting the costs inherent to run them. However, as the integration of the MEC increases the reaction rates, a different KPI focused purely on the methane productivity seems more appropriate.

Therefore, for the comparison between the ModIn and ModEx configurations against the AD control, the methane production rate (MPR, in ml_{CH4}/L day) is preferred as a KPI that demonstrates the methane productivity in terms of volume of methane per unit of reactor volume and time, which has a direct interpretation to the industrial scale. To avoid the influence of the transient period of increasing methane production rate in the comparison, it is selected the last month of operation, that represents the steady performance of the bioreactors after the initial start-up stage.

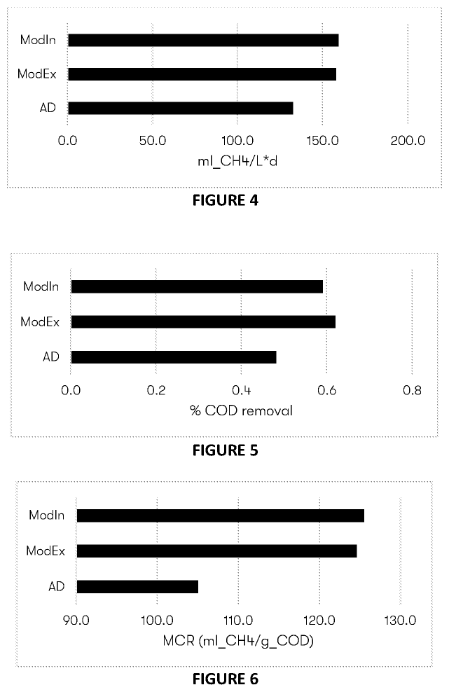

FIG. 4 shows the average Methane production rate in ml_{CH4}/L day. This data is also shown in Table 3, along with the standard deviation and the coefficient of variation.

TABLE 3
Average Methane production rate per configuration, including
data between 27 Aug. 2022 and 20 Sep. 2022

| Row | Average of | StdDev of | Coeff of |
|---|---|---|---|
| Labels | MPR [ml_{CH4}/L*d] | MPR [ml_{CH4}/L*d] | variation |
| AD | 132.5 | 17.6 | 13% |
| ModEx | 157.9 | 3.7 | 2% |
| ModIn | 159.3 | 13.3 | 8% |

Clearly, the MEC reactor, either internal or in an external module, produces an improvement of about 20% when compared to the AD control. Importantly, while the bulk of the reactant liquor is within the chamber of the reactor and remote from the MEC electrodes, the ModEx configuration mirrors the performance of ModIn. This validates the ModEx integration strategy to retrofit the technology into existing anaerobic digesters.

The coefficient of variation shows the relative weight of the standard deviation compared to the average, and suggests a high standard deviation when >1, and low when <1. A single factor ANOVA was performed to evaluate whether the improvement of the overall performance for the ModIn and ModEx configurations is statistically significant. This established that both ModIn and ModEx shown a significantly different performance against the AD, but there is no significant difference between the performance of ModIn and ModEx configurations (FIG. 4).

### COD Removal Performance

COD removal efficiency was calculated from the raw data according to the equation:

$${COD}_{removal efficiency} = F * \frac{( {COD}_{in} - {COD}_{out} )}{{COD}_{in}}$$

With this parameter and considering all the data available (see Appendix C), except those reactors that had operational failures at some point, the average of the same period used for the MPR comparison is calculated as shown in Table 4 and FIG. 5. The COD removal was slightly improved in the ModEx and ModIn configurations, compared to the AD control.

TABLE 4
Chemical Oxygen Demand removal efficiency per configuration,
including data between 27 Aug. 2022 and 20 Sep. 2022

| Row | Average % | StdDev of MPR | Coeff of |
|---|---|---|---|
| Labels | COD removal | [ml_{CH4}/L*d] | variation |
| AD | 0.5 | 0.1 | 14% |
| ModEx | 0.6 | 0.09 | 16% |
| ModIn | 0.6 | 0.02 | 3% |

Anaerobic digestion links the energy matrix and the organic waste network; therefore, it can therefore be applied as either an energy production or waste disposal technology. The latter is a more common application for anaerobic digestion due to the traditionally low reaction rates. For a waste treatment, COD removal is the most relevant performance parameter.

Since the present invention is directed to energy production, not waste disposal, the improvement in COD removal is noteworthy. It is an indirect consequence of the carbon deviation towards the off gas instead of cellular growth, and an additional benefit provided by the present invention. This effect can also be noticed in the increment of the methane conversion rate (MCR, in ml_{CH4}/g_{COD}) that corresponds to the yield of methane per unit of COD fed. Table 5 and FIG. 6 summarise the performance for the ModIn and ModEx configurations compared with the AD control.

TABLE 5
MCR per configuration versus the AD control, including
data between 27 Aug. 2022 and 20 Sep. 2022

| Row | Average of MCR | StdDev of MPR | Coeff of |
|---|---|---|---|
| Labels | [ml_{CH4}/g_{COD}] | [ml_{CH4}/L*d] | variation |
| AD | 105.0 | 27.0 | 26% |
| ModEx | 124.6 | 2.2 | 2% |
| ModIn | 125.5 | 10.2 | 8% |

### Example 4—Pre-Anodisation

### Materials and Methods

The analysis was carried with a 3-electrode, 100 ml single chamber electrochemical cell. The cell had a working electrode (anode) of 3×1 cm of carbon graphite felt; titanium wire as a pseudo-reference electrode; and a counter electrode being a 6 mm Stainless steel (SS) tube, separated 2 cm apart from the working electrode. The electrolyte used was 100 ml of 10 g/L FeSO₄ solution.

Pre-anodisation of the working electrode was carried out by applying a voltage of 1.5 V to an anode of graphite felt, in cycles of 15 mins On/Off. This was repeated for 24 hours.

### Methodology Overview

In porous electrode materials, it is very hard to estimate the actual surface available for electrochemical reactions. However as a source of comparison, graphite electrodes of different total surface areas are used to construct a curve where peak current and charge are correlated to the corresponding surface area, allowing use of a regression equation to estimate the “sample” material surface area from the obtained peak current/charge.

This analysis allows estimation of the actual surface area of the electrodes, and also enables comparison of the effect of some pre-treatment parameters such as the number of anodisation cycles, and electrolyte strength used. The assessment procedure has three steps:
Build a reference graph of peak current against known surface electrodes areas
Make regression, ideally linear at various scan rates to chose the best fit (avoiding diffusion limitations)
determine peaks current of unknown sample to estimate the surface area.

### Peak Current Method

This method allows measurement of the electroactive surface area of a porous electrode, based on the linear relation between peak current and the electroactive surface area. The electroactive surface area measured by this method can be affected by the particle size, sintering temperature and/or chemical etching of the electrode if any, in addition to pore size and porosity. It is particularly sensitive to the diffusion layer thickness, which is a function of CV scan rate. The steps are:
Build a surface area vs peak current plot for flat plate-like electrodes of known surface area (different) to obtain the linear relationship between them;
By performing a series of voltammograms, identify whether there is more than 1 reaction taking place and select that controlled solely by the ion diffusion;
Repeat the process at various scan rates, to build a plot peak current vs surface area, where various straight lines can be drawn with the different scan rates;
If the lines are straight, the system responds to the Randles-Sevcik equation.

### Results

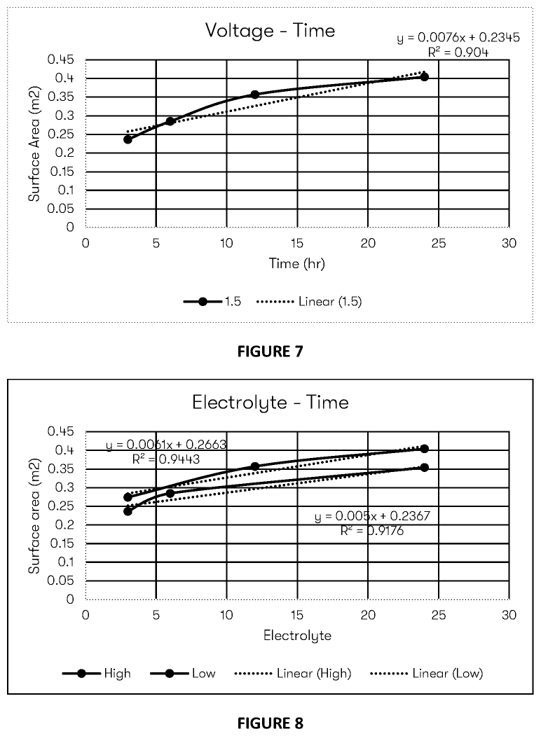

The voltage increases over time when cycles of 15 mins On/Off of 1.5 V are applied over a 24-hour period to an anode of graphite felt (FIG. 7), indicating an increase in the surface area of the electrode.

As seen, there is a clear positive correlation between the duration of voltage cycling and the measured surface area. Here the actual surface area in m² of the pieces of treated electrode is reported, the electrode was 1×3 cm with a 3 mm thickness. It can be seen the surface area is increased by about 64% within 24 hours of treatment from 0.23 to 0.41 m².

Two different electrolyte strengths were compared, at 4.3 mg/L and 430 mg/L of Fe₂SO₄. Although the low-strength electrolyte seems to achieve greater surface areas, the increment achieved is not significant, at 33% compared to the 32% achieved by the high strength electrolyte. Both electrolytes also show a similar trend in terms of the slope of the surface area increment over time of voltage cycling (FIG. 8). This indicates a similar increase in surface area can be achieved using either a low-strength electrolyte or a high-strength electrolyte. The person skilled in the art will recognize that there are a number of salts suitable for use as an electrolyte to increase the surface area of the electrode

This difference on the surface estimation of the two electrolyte strengths, is probably due to the impact of the double layer thickness formed during the process, where the system may work as a capacitor storing more or less energy, affecting the peak current measurement.

### Example 5—Microbial Attachment

### Materials and Methods

A three-electrode cell including a WE (working electrode), CE (counter electrode) and a reference electrode (RE), was used to control the potential of one specific electrode, in this case the cathode, which for the microbial attachment/colonisation stage, was connected as anode. During the microbial attachment stage, the anodic potential attracts electroactive microorganisms such as methanogens which would not be attracted to a cathode.

A four-electrode cell was be used instead of the three-electrode cell to prepare some samples. The four-electrode cell accommodates a second WE, arranging the distance between each WE and the CE and RE respectively to be as similar as possible. This allows testing of different materials as WEs, in this case GF and pre-anodised GF, under the same conditions.

The Counter electrode (CE) used was 6 mm×2 cm stainless steel tube (316L) having thickness of about 0.1 mm, which offers good electrical conditions and is not expected to be colonized by microorganisms. Due to the complexity of the manure waste as electrolyte, and the complexity of bacterial growth in the presence of solids, a typical electrode reference such as Ag/AgCl could suffer significant variation as the frit of the electrode may clog. Therefore a pseudo-reference electrode (RE) of coiled titanium wire is used in this example. Working electrodes (WE) tested were of dimensions 1×3 cm, and included with graphite felt (GF) and pre-anodised GF (thickness of 3 mm).

Once the cell was set up, a potential of about +1.5 V/SCE was applied to the WE for a period of 8 hours to encourage attachment of methanogenic bacteria.

The development of a biofilm on the electrodes can be monitored by collecting cyclic voltammograms that indicate how much charge is being transferred, peak current and other information as described below. Changes in the WE voltammogram over time can indicate surface modifications that can be attributed to microbial attachment given the context of the test.

The described test used both a microbial consortium and a nutrient medium. The nutrient medium was a yeast extract medium (2 g/L yeast extract+20 ml/L of salts), and the microbial consortium was introduced using a settled 1:7 diluted manure. WEs were monitored regularly through cyclic voltammetry runs at 0.15 V/sec between −4 and 4 volts, starting at 0.1V on steps of 0.02V. The voltammograms were performed: initially, for the nutrient medium; after manure was added as inoculum; and then every 1-2 hours for a total period of 8 hours of experiment; the upper time limit being chosen to avoid the duplication of any archaea or bacteria involved in the process of methane production.

The voltammograms were monitored, and compared according to peak current, the ratio Iₐₙ/I_{cat}, and the potential of I_{cat}.

To assess whether the materials and pre-treatment have any measurable effect over the colonization of the electrodes, the WE-CE electrode pairs were tested individually following the steps:
Place the electrodes in a three-electrode configuration electrochemical cell;
Use 50 ml (about 50% of the cell capacity) of electrolyte (nutrient medium without another source of COD);
Perform a first cyclic voltammetry analysis;
Add COD source to a final volume of about 50% of the cell capacity and final COD desired;
Perform a new cyclic voltammetry analysis under identical CV parameters as the first cyclic voltammetry analysis;
Add inoculum;
Perform a further two CV analysis under identical CV parameters as the first cyclic voltammetry analysis every 1-2 hours.

### Microscopy

UV Fluorescence microscopy was performed on multiple fresh samples of GF and pre-anodized GF electrodes, which had been prepared by applying a 1 V anodic potential to the working electrode for three hours, in the presence of 50% v/v feedstock manure +50% synthetic municipal wastewater. Each sample was air-dried before a fluorescent DAPI stain was applied. Multiple fields were imaged, to assess homogeneity and establish a number of fields required for assessing future samples.

### Results

### Microscopy

An estimate of the percentage area of the electrode covered by bacteria was obtained using DAPI staining and fluorescence microscopy (Axio Imager M2, Zeiss) at 400× magnification. Some issues with the methodology for estimating the area included the presence of a heavily stained area in the middle of the anodized samples, and some autofluorescent fibres. For this reason the values for the anodized samples were obtained from the edge of the sample. The heavily stained fluorescence area may be a patch of organic debris present in the manure that got stuck into the material during the process, for example fat that has been reported to produce unspecific DAPI staining, and it is clearly seen as a thin upper layer in the manure, which could have attached when removing the electrode from the manure.

The estimation of the percentage area covered by bacteria from the microscopy results was 4.34% for the GF sample, and 3.38% for the anodized sample.

### Cyclic Voltammetry

For the anodised GF samples it was observed that the anodic current peak is reduced as time progresses, with a bigger change in the anodic current peak during the first 30 minutes, compared to the value at 0 minutes.

For the GF samples, the shape of the voltammograms are slightly different, as the anodic current peak is not seen, and the obtained currents are also lower than those obtained for the anodised GF samples (3500 vs 5500 μA).

The drop in the peak current observed corresponds to about 4-5%, very close to the approximate surface area covered of 3.38-4.34% according to the microscopy analysis.

Due to the similarity of the voltammograms in terms of peak location and change in current values over time, the inventor concludes that the variation in the voltammogram for either the GF sample or the anodized GF sample is due to a surface modification, which can be understood to be a consequence of the surface colonization observed in microscopy analysis.

### Example 6—Effect of Voltage, Electrode Materials

### Materials and Methods

The effect of voltage on the reactors used in Example 3 was investigated as follows. Using a full cell potential control strategy, three different conditions were used for each of the AD, ModEx, and ModIn configurations: open circuit potential (OCP), 0.8V and 1.5 V. The steady state operation had 1.3 g_{COD}/L*D as organic loading rate, 20 days of HRT, and 38 °C as the temperature set point. Two different materials were investigated for the cathodes: graphite felt (GF) and stainless steel mesh (SS). GF electrodes were used for all anodes.

The reactors were operated at 0.8 V until steady state, then changed to OCP or 1.5V, until the steady state has been maintained for about 2 months. A series of 24 parameters were monitored as system health indicators. The most relevant performance indicator was found to be the MPR in LCH₄/m³*d. This KPI is selected due to the significance on the volumetric productivity of methane of the bioreactor.

### Results

The results are shown in Table 6.

TABLE 6

| | | | Biogas | | MPR | |
|---|---|---|---|---|---|---|
| Voltage | | Cathode | Production | % | (L_{CH4}/m³ | % |
| (V) | Reactor | material | (L/m³*d) | Improv | (reactor)*d) | Improv |
| OCP | Control | N/A | 233 | 0% | 230.87 | 0% |
| | AD | | | | | |
| | ModEx | GF | 270 | 16% | 269.09 | 17% |
| | | SS | 257 | 11% | 241.14 | 4% |
| | Mod In | GF | 231 | −1% | 240.45 | 4% |
| | | SS | 245 | 5% | 237.05 | 3% |
| 0.8 | ModEx | GF | 303 | 30% | 304.77 | 32% |
| | | SS | 285 | 23% | 278.35 | 21% |
| | Mod In | GF | 294 | 26% | 308.96 | 34% |
| | | SS | 300 | 29% | 308.57 | 34% |
| 1.5 | ModEx | GF | 259 | 11% | 250.94 | 9% |
| | | SS | 262 | 13% | 265.52 | 15% |
| | Mod In | GF | 236 | 2% | 261.38 | 13% |
| | | SS | 245 | 5% | 263.2 | 14% |

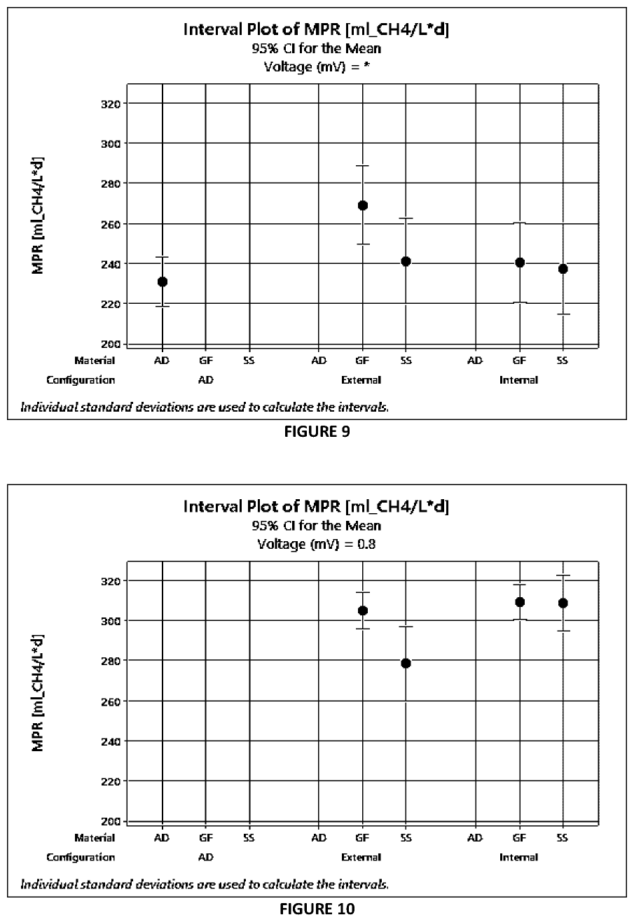

Since there was no mechanism to impose different voltages on the AD reactor, the AD reactor is only shown on the OCP control (FIG. 9).

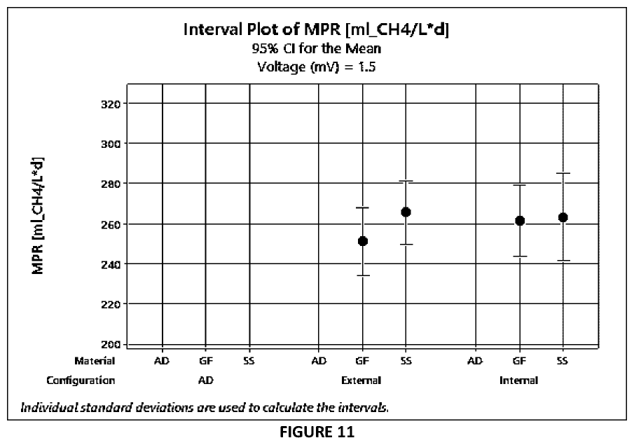

At first glance, it is noticeable how the OCP performance of the ModIn and ModEx reactors tend to be very close to that of the Control AD (Table 6, FIG. 9), merely a 3 or 4% in terms of MPR, despite the 11 or 16% achieved in terms of volumetric productivity of biogas. This suggests a decrease on the methane content of the biogas produced. However, when comparing a reactor having a SS cathode to a reactor having a GF cathode, the GF cathode clearly leads to a significant improvement in the MPR. This suggests that the GF (still present in the anode of every prototype) effectively aids the hydrolysis stage (considered here as the bottleneck of the AD process for cow manure). Also surprisingly, applying a voltage of 1.5 V does not lead to greater performance than 0.8 V (compare FIGS. 10 and 11), so the preferred voltage for these reactors is between these two values. The biogas production increases between 23 and 30% when 0.8 V are applied, but only 2 to 13% when 1.5 V are used. It is noteworthy here that there is virtually no difference per configuration when 0.8 V are used, but at 1.5 V, ModEx only produces 2 to 5% of additional biogas production.

The better performance is more striking in terms of MPR; while applied voltage of 1.5 V causes an increase in MPR of between 9 and 15%, applied voltage of 0.8 V causes an increase in MPR of between 21 and 34%. Although, ModEx-SS is the only below 30% at 0.8 V, in fact both ModIn reactors achieved a 34% improvement in MPR, regardless of the electrode material, which again, suggests that the main effect for cow manure is on the anode rather than the cathode.

It is not the intention to limit the scope of the invention to the abovementioned examples only. As would be appreciated by a skilled person in the art, many variations are possible without departing from the scope of the invention as set out in the appended claims.

1. A bioelectrode comprising a bacterial biofilm, wherein the bioelectrode has a porous structure with a range of pore sizes.

2. The bioelectrode according to claim 1, wherein the bioelectrode comprises primary pores in a first size range, and secondary pores in a second size range.

3. The bioelectrode according to claim 1, wherein the bioelectrode comprises a biocompatible material and/or a carbon-based material, for example felts, papers, sintered, porous material of either carbon, metallic or plastic base.

4. The bioelectrode according to claim 1, wherein the electrode comprises strands of fibres.

5. The bioelectrode according to claim 4, wherein the fibres form a 3D open structure of primary pores.

6. The bioelectrode according to claim 4, wherein the secondary pores are provided as indentations.

7. The bioelectrode according to claim 6, wherein the indentations are formed by a pre-treatment of the fibres.

8. The bioelectrode according to claim 7, wherein the pre-treatment comprises applying a potential to the electrode.

9. The bioelectrode according to claim 1, wherein the electrode surface is functionalised by immobilized microbes, enzymes, or a catalytic material.

10. The bioelectrode according to claim 1, wherein the electrode has a surface area of at least 1 m²/g, more preferably at least 2 m²/g.

11. The bioelectrode according to claim 1, wherein the biofilm is a mature biofilm.

12. The bioelectrode according to claim 1, wherein the biofilm comprises at least one methanogenic microorganism; and at least one electroactive microorganism.

13. A microbial electrolysis cell (MEC) comprising a solid-state cathode; a solid-state anode; and an electro-active biofilm.

14. The MEC according to claim 13, which comprises a bioelectrode comprising a bacterial biofilm, wherein the bioelectrode has a porous structure with a range of pore sizes.

15. The MEC according to claim 13, further comprising a dielectric material between the cathode and the anode.

16. A process for colonizing a bioelectrode with microorganisms, comprising steps of
contacting an electrode with a mixture comprising microorganisms, and
applying a potential to the electrode during the contacting step.

17. The process according to claim 16, wherein the potential applied during the contacting step is about +1.5 V/SCE.

18. The process according to claim 16, wherein the potential is applied during the contacting step for a period of at least about 1 hour, preferably at least about 2 hours, more preferably for about 2 hours.

19. The process according to claim 16, wherein the potential applied during the contacting step is an anodic potential, and the microorganisms which colonize the bioelectrode comprise methanogenic microorganisms.

20. The process according to claim 16, wherein the process further comprises a pre-anodisation step of applying an anodic potential to the electrode prior to the contacting step.

21. The process according to claim 20, wherein the pre-anodisation step comprises applying a potential of about +1.5 V/SCE.

22. The process according to claim 20, wherein the potential is applied to the electrode in cycles during the pre-anodisation step.

23. The process according to claim 22, wherein the cycles are of a period of about 15 min on, 15 min off.

24. The process according to claim 22, wherein successive cycles are applied over a period of about 24 hours.

25. A process for anaerobic digestion of a feedstock in an anaerobic digester, the process comprising:
providing a feedstock to a chamber of anaerobic digester to form a reactant liquor in the anaerobic digester;
contacting the reactant liquor with a bioelectrode of a microbial electrolysis cell which comprises the bioelectrode and a counter electrode, and
applying a potential to the reactant liquor.

26. The process according to claim 25, wherein the bioelectrode of the microbial electrolysis cell is positioned within the chamber of the anaerobic digester.

27. The process according to claim 25, wherein the bioelectrode comprises a biofilm.

28. The process according to claim 25, wherein the bioelectrode comprises a bacterial biofilm, and wherein the bioelectrode has a porous structure with a range of pore sizes.

29. The process according to claim 25, wherein the bioelectrode is a cathode.

30. The process according to claim 29, wherein the cathode is adapted to favour direct methanogenesis.

31. The process according to claim 29, wherein the cathode is adapted to favour direct interspecies electron transfer.

32. The process according to claim 29, wherein the cathode comprises graphite felt.

33. The process according to claim 29, wherein the cathode comprises stainless steel.

34. The process according to claim 25, wherein the bioelectrode is separated from the counter electrode by a dielectric material.

35. The process according to claim 25, wherein the anaerobic digester comprises a microbial consortium comprising planktonic microbes and the bioelectrode of the microbial electrolysis cell comprises a microbial biofilm.

36. The process according to claim 25, wherein the microbial electrolysis cell is positioned inside the chamber of the anaerobic digester.

37. The process according to claim 25, wherein the microbial electrolysis cell is positioned outside the chamber of the anaerobic digester, and is in fluid communication with the chamber of the anaerobic digester.

38. The process according to claim 25, wherein the voltage applied between the bioelectrode and the counter electrode is greater than 100 mV, or greater than 300 mV, or greater than 600 mV.

39. The process according to claim 25, wherein the voltage applied between the bioelectrode and the counter electrode is in the range 600 mV to 1200 mV, preferably in the range 800 mV to 1000 mV.

40. The process according to claim 25, wherein the temperature in the anaerobic digester is within the range from about 30 to about 45 °C, preferably from about 39 to about 42 °C, or from about 35 to about 39 °C, more preferably about 38 °C.

41. The process according to claim 25, wherein the feedstock/reactant liquor comprises municipal wastewater and/or manure, preferably with a total solids content between 1 and 10%, more preferably between 1 to 5% or 5 to 7%.

42. An external module comprising a chamber, wherein the chamber is adapted for fluid communication with a chamber of an anaerobic digester.

43. The external module according to claim 42, wherein the chamber is adapted to receive a bioelectrode comprising a bacterial biofilm, wherein the bioelectrode has a porous structure with a range of pore sizes.

44. The external module according to claim 42, wherein the chamber further comprises a bioelectrode comprising a bacterial biofilm, wherein the bioelectrode has a porous structure with a range of pore sizes.

45. The external module according to claim 44, wherein the bioelectrode was produced by a process for colonizing a bioelectrode with microorganisms, comprising steps of: contacting an electrode with a mixture comprising microorganisms, and applying a potential to the electrode during the contacting step.

46. The external module according to claim 42, further comprising a pump for exchanging fluid between the chamber of the external module and the chamber of the anaerobic digester.

47. The external module according to claim 42, wherein the external module is adapted to be stackable with another external module.

48. The anaerobic digester comprising a bioelectrode according to claim 1.

49. The anaerobic digester according to claim 48, wherein the bioelectrode was produced by a process for colonizing a bioelectrode with microorganisms, comprising steps of: contacting an electrode with a mixture comprising microorganisms, and applying a potential to the electrode during the contacting step.
